(19) United States
(12) Patent Application Publication
(10) Pub. No.: US 2005/0216006 A1
(43) Pub. Date: Sep. 29, 2005

(54) Cap for cut metal orthopedic fastener

(76) Inventors: Jorge L. Orbay, Miami, FL (US); Javier Castaneda, Miami, FL (US)

Correspondence Address: Gordon & Jacobson, P.C., 65 Woods End Road, Stamford, CT, 06905, US

(21) Appl. No.: 10/806,540

(22) Filed: Mar. 23, 2004

(51) Int. Cl. A61B 17/72

(52) U.S. Cl. 606/62

(57) ABSTRACT

A preferably radiopaque plastic cap is provided which is sized and shaped to facilitate placement over the cut end of a K-wire, orthopedic nail, or similar shaped device, and for frictional engagement on the device. The plastic cap completely covers the non-passivated portion of the cut metal, thereby eliminating or substantially reducing the potential for negative reaction by the surrounding tissue. Moreover, there is reduced pain to the patient when flexing muscles surrounding the ends of the fasteners, and the skin can slide over the cap without irritation or inflammation.

### BACKGROUND OF THE INVENTION

1. Field of the Invention

This invention relates broadly to orthopedic fasteners. More particularly, this invention relates to protective caps for orthopedic fasteners.

2. State of the Art

In various orthopedic procedures, it is common to cut a stainless steel wire or nail, and seat the cut end of the wire or nail under the skin for later removal after bone healing. However, the cut end of the wire or nail can be reactive. This is because, unlike the remainder of the wire or nail, the cut end has not been passivated. Furthermore, the cut end is rough and has a relatively high surface area. These factors combine to cause a reaction with the surrounding tissue and blood, and may result in the formation of rice bodies, which generally does not occur at such locations in the presence of non-cut fasteners. Rice bodies may contain coarse collagenous fibers, reticulin and elastin which vary in consistency, size and shape, but which resemble grains of rice.

Furthermore, it is desirable to facilitate location of subcutaneous wires or nails after bone healing to facilitate removal thereof.

### SUMMARY OF THE INVENTION

It is therefore an object of the invention to provide a system to provide a means for reducing contact between non-passivated metal and tissue.

It is another object of the invention to provide a means for reducing contact between unfinished, sharp, and/or rough metal and tissue.

It is a further object of the invention to provide a means for easily identifying the location of subcutaneous wires or nails.

It is also an object of the invention to provide a cover which is particularly suitable for use over the cut end of a K-wire or orthopedic nail.

In accord with these objects, which will be discussed in detail below, a plastic cap is provided which is sized and shaped to facilitate placement over the cut end of a K-wire, orthopedic nail, or similar shaped device, and for frictional engagement on the device. The plastic cap completely covers the non-passivated portion of the cut metal, thereby eliminating or substantially reducing the potential for negative reaction by the surrounding tissue. Moreover, there is reduced pain to the patient when flexing muscles surrounding the ends of the fasteners, and the skin can slide over the cap without irritation or inflammation.

According to a preferred aspect of the invention, the cap is radiopaque. The radiopacity allows the surgeon to verify the precise location of the end of the wire, nail, or device under fluoroscopy and also ensure the device is fully seated.

Additional objects and advantages of the invention will become apparent to those skilled in the art upon reference to the detailed description taken in conjunction with the provided figures.

### BRIEF DESCRIPTION OF THE DRAWINGS

FIG. 1 is a perspective view showing the cap of the invention coupled over the end of a cut wire, nail or similar device;

FIG. 2 is a side elevation similar to FIG. 1;

FIG. 3 is a side elevation of an embodiment of the cap of the invention; and

FIG. 4 is a section view across line 4-4 in FIG. 3.

### DETAILED DESCRIPTION OF THE PREFERRED EMBODIMENTS

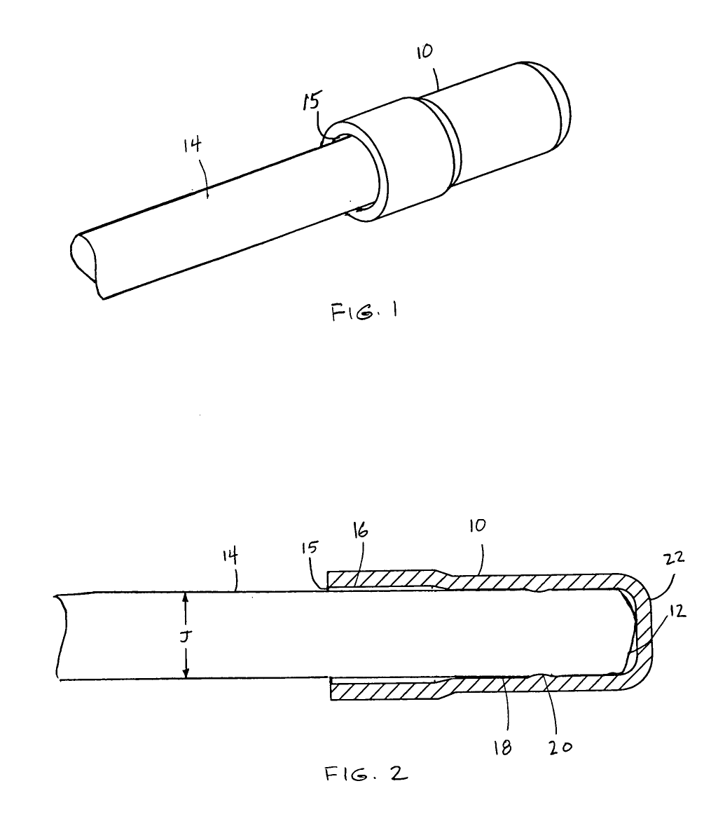

Turning now to FIGS. 1 and 2, the cap 10 of the invention is shown coupled over the cut end 12 of a nail 14, wire, or similar substantially cylindrical device. By way of example, and not by limitation, a similar device is the locking device described in U.S. Pat. No. 6,533,788, which is hereby incorporated by reference herein in its entirety. For convenience, hereafter in the description and claims, the term “nail” shall be considered to include any of the above and similar devices which are implanted into bone, cut and seated beneath the skin.

The cap 10 is preferably made from a biocompatible, lubricious and resilient plastic, such as polyurethane. In accord with a preferred aspect of the invention, the plastic is preferably embedded with a radiopaque material, such as barium. In a preferred formulation, forty percent barium sulfate is provided in the plastic to effect the radiopacity. This permits the cap to be visualized under fluoroscopy so that the surgeon can verify the precise location of the end of the nail under fluoroscopy and also ensure the nail is fully seated.

In general, the cap 10 includes a lead-in (or entry) portion 16 which is slightly larger than the outer diameter of the nail 14 so as to provide a clearance 15 which facilitates entry of the cut end 12 of the nail into the opening of the cap. The remainder of the cap defines an engagement portion 18 preferably designed to have a slight interference with the outer diameter of the nail 14 so that some retaining engagement is provided therebetween. Within this tighter engagement portion 18, there is a protruding ring 20 with a preferably convex cross-section that preferably provides a majority of the friction which prevents migration of the cap 10 on the end of the nail 14. As an alternative to an unbroken ring 20, a single or plurality of spaced-apart protuberances may be circumferentially located about the inner wall of the engagement portion. The engagement portion 18 is substantially longer than the lead-in portion 16. Both the lead-in portion 16 and engagement portion 18 are substantially cylindrical tubular portions, with the lead-in portion 16 tapering in dimension to the engagement portion 18. Alternatively, each of the tubular portions may be defined by, e.g., five or more sides so as approximate a cylindrical tube. While such may not be as circumferentially smooth (for reduced tissue irritation), the cap 10 will have greater tactile sensation to the surgeon facilitating its implantation on the cut end of the nail. The cap 10 includes a closed end 22. Thus, the cap 10 facilitates easy entry, but secure and protective seating on the end of the nail 14 in a small size protective device.

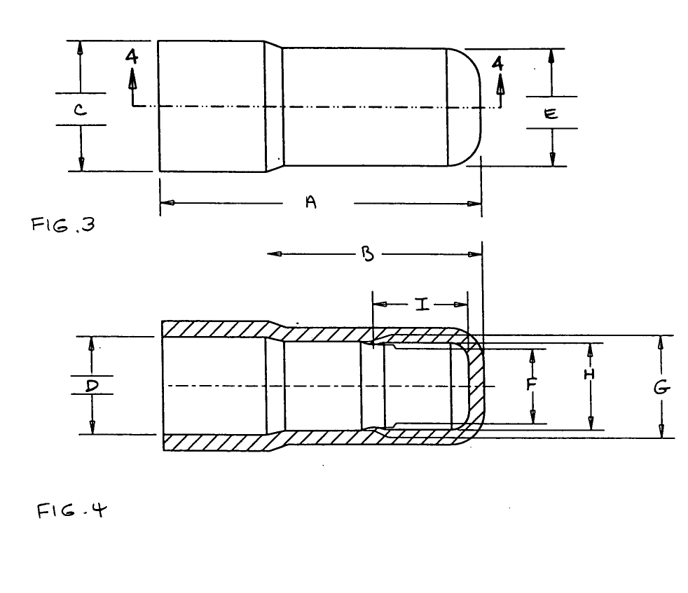

Referring to FIGS. 3 and 4, exemplar dimensions are provided for a preferred embodiment of the cap. The cap 10 preferably has a length A of 0.285-0.310 inch, with an engaging length B of approximately 0.20 inch. The lead-in portion 16 has an outer diameter C of approximately 0.109-0.122 inch, and an inner diameter D of approximately 0.080-0.092 inch. The engagement portion 18 has an outer diameter E of approximately 0.098-0.110 inch. The convex ring 20 defines an inner diameter F of approximately 0.067-0.078 inch. The engagement portion 18 has an inner diameter G of approximately 0.072-0.084 inch distal of the convex ring 20 (toward the lead-in portion 16) and a slightly smaller diameter H proximal of the ring 20 (toward the closed end 22), e.g., smaller by approximately 0.002 inch. This is because the proximal portion of the engagement portion 18 will be stretched in diameter as the nail contacts the ring 20. The length I of the engagement portion 18 proximal of the ring 20 is approximately 0.09 inch or about two-thirds the total length of the cap. The exemplar dimensions are particularly desirable for a nail having an outer diameter J (FIG. 2) corresponding to inner diameter F (approximately 0.072-0.084 inch), facilitating placement over the cut end 12, and enabling secure seating on the nail 14 by way of frictional engagement.

There has been described and illustrated herein an embodiment of a radiopaque cap for a cut end of an orthopedic nail. While a particular embodiment of the invention has been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its scope as claimed.

1. A cap for a cut end of a nail, comprising:
a) a lead-in tubular portion having an inner surface which defines a first diameter;
b) an engagement tubular portion having an inner surface which defines a second diameter smaller than said first diameter; and
c) a closed end at a proximal end of said engagement portion,
wherein said cap is manufactured from a plastic radiopaque material.

2. A cap according to claim 1, wherein:
said plastic is resilient.

3. A cap according to claim 2, wherein:
said plastic is polyurethane.

4. A cap according to claim 1, further comprising:
d) an inner protuberance provided along said inner surface of said engagement portion.

5. A cap according to claim 1, wherein:
said lead-in portion is cylindrical.

6. A cap according to claim 5, wherein:
said engagement portion is cylindrical.

7. A cap according to claim 6, wherein:
said engagement portion is longer than said lead-in portion.

8. A cap according to claim 1, wherein:
said lead-in portion defines an inner diameter of approximately 0.080-0.092 inch.

9. A cap according to claim 1, wherein:
said engagement portion defines an inner diameter of approximately 0.070-0.084 inch.

10. A cap according to claim 1, wherein:
said cap has a length of approximately 0.285-0.310 inch.

11. A cap for a cut end of a nail, comprising:
a) a lead-in tubular portion having an inner surface which defines a first diameter;
b) an engagement tubular portion having an inner surface which defines a second diameter smaller than said first diameter;
c) an inner protuberance provided along said inner surface of said engagement portion and directed substantially radially inward; and
d) a closed end opposite said lead-in portion.

12. A cap according to claim 11, wherein:
said cap is manufactured from a plastic radiopaque material.

13. A cap according to claim 11, wherein:
said inner protuberance is a ring.

14. A cap according to claim 13, wherein:
said ring has a convex outer surface.

15. A cap according to claim 11, wherein:
said engagement portion has an inner surface defining a third diameter smaller than said second diameter, wherein said second diameter is provided distal of said protuberance and said third diameter is provided proximal of said protuberance.

16. A cap according to claim 11, wherein:
said lead-in portion has an inner diameter of approximately 0.080-0.092 inch.

17. A cap according to claim 11, wherein:
said engagement portion has an inner diameter of approximately 0.070-0.084 inch.

18. A cap according to claim 1 1, wherein:
said cap has a length of approximately 0.285-0.310 inch.

19. A cap for a cut end of a nail, comprising:
a resilient plastic cylindrically tubular element having a closed end and an inner surface, said tubular element having an inner diameter sized to be close fitting to the nail, a length at least approximately three times said inner diameter, and including an inner protuberance provided along said inner surface which frictionally engages the nail.

20. A cap according to claim 19, wherein
said plastic includes a radiopaque material.

21. A combination orthopedic nail and cap therefor, comprising:
a) a metal orthopedic nail having an end with a first diameter; and
b) a cap provided over said end of said nail,
said cap including a lead-in portion having an inner surface which defines a second diameter larger than said first diameter such that said lead-in portion is spaced apart from said end of said nail by a clearance, an engagement portion having an inner surface which defines a third diameter approximating said first diameter such that said engagement portion is substantially in contact with said nail, and a closed end.

22. A combination according to claim 21, wherein:
said end of said nail is a non-passivated cut end.

23. A combination according to claim 21, wherein:
said cap is manufactured from a plastic radiopaque material.

24. A combination orthopedic nail and cap therefor, comprising:
a) a metal orthopedic nail having a first diameter and a cut end; and
b) a radiopaque cap provided over said cut end of said nail,
said cap including a plastic cylindrically tubular element having a closed end and an inner surface, said tubular element having an inner second diameter sized to be close fitting to the nail, a length at least approximately three times said second diameter.

25. A method of covering an end of a metal orthopedic nail, comprising:
a) implanting the orthopedic nail into a bone, said nail having an exposed end;
b) providing a radiopaque plastic cap over the end of the nail; and
c) seating the end of the nail and the cap beneath the skin.

26. A method according to claim 25, further comprising:
prior to placing the cap, cutting the exposed end of the nail such that providing the cap over the end of the nail provides the cap over a cut end of the nail.
